(19) United States
(12) Patent Application Publication
(10) Pub. No.: US 2011/0027199 A1
(43) Pub. Date: Feb. 3, 2011

(54) Drink container with a breath strip

(76) Inventors: Danny Frye, Fletcher, NC (US); Jason Reagan, Fletcher, NC (US)

Correspondence Address: Patent Consultants & Services, Inc., 204 Bouchillion Drive, Greenville, SC, 29615, US

(21) Appl. No.: 12/802,955

(22) Filed: Jun. 17, 2010

Related U.S. Application Data
(60) Provisional application No. 61/273,076, filed on Jul. 30, 2009.

(51) Int. Cl. A61K 8/365; B65D 25/00; B65D 25/34; A61K 8/34; A61Q 11/00

(52) U.S. Cl. 424/55; 206/217; 220/738; 424/49

(57) ABSTRACT

The present invention by provides a system for treating the symptoms of a user having a bad breath or taste after drinking a liquid substance. The invention comprises a drink container initially containing the liquid substance consumed by the user. A breath strip assembly forming a strip pouch is carried by the drink container. A single soluble film breath strip is housed in the strip pouch to be accessed by the user and dissolved in the mouth to remove the users bad breath and taste. In one aspect the breath strip assembly comprises a flexible substrate having front and rear surfaces. The rear surface is attached to the drink container using a pressure sensitive rear adhesive between the rear surface and the drink container. A removable film is partially attached to the front surface of the substrate using a pressure sensitive front adhesive on the substrate in a pattern to provide the strip pouch.

### RELATED APPLICATIONS

This application claims priority to U.S. Provisional Patent Application 61/273,076 filed Jul. 30, 2009, the entireties of which are hereby incorporated by reference.

### BACKGROUND OF THE INVENTION

This invention is directed to providing a drink container with a breath strip assembly attached to the container to be accessed by the user after drinking. The breath strip is available from a pouch of the assembly for killing the plaque-producing germs that cause dental plaque, gingivitis and especially bad breath and bad taste as a result of consuming the liquid substance of the drink.

A number of beverages that are consumed from a hand held drink container cause the drinking person to acquire a breath which is objectionable to people near the drinking person. The beverage may also result in the consumer having a bad taste in their mouth. For example, beverages that can cause an objectionable breath or taste include coffee, milk and some fruit juices. Most people will have some kind of mint, breath strip or cough drop that can be dissolved in the mouth to kill the germs and create an instant fresh breath. However, most people do not have these items readily available when needed. Women may carry something in their purse but cannot locate them; and men are not likely to carry anything in their pockets. A need exists to have a breath strip readily available when the bad breath or taste is first initiated. Bad breath or bad taste is collectively referred to herein as a bad breath. Soluble film breath strips are well known in the industry. These edible films are effective as killing the plaque-producing germs that cause dental plaque, gingivitis and bad breath. Edible films include pullulan and antimierobially effective amounts of the essential oils thymol, methyl calicylate, encalyptol and menthol. Orally consumable films are disclosed in U.S. Pat. Nos. 6,596,298; 6,669,020; 6,776,284; 6,923,981; and 7,025,983. These edible films contain the necessary ingredients to kill the germs in the mouth for an instant fresh breath.

Breath strips are commonly available over-the-counter to the public in some type of small container or package. The package can be carried in a pocket or purse for easy access by the user. Typical blister packages or cases are disclosed in U.S. Pat. Nos. 6,669,020; 6,726,054; 6,776,284; 7,104,419; 7,165,676; 7,357,255; 7,434,692; and 7,506,762. The blister packages generally have moisture barrier and heat resistant properties with a number of soluble films in each package that are accessed one at a time and with the remaining films resealed in the package. For example, the PocketPaks® by LISTERINE® have 24 breath strips in each packet. The question is, where is the packet when you need it? The need exists for a single breath strip which is immediately available when needed.

Edible films have also been used to providing medications. There are film-forming materials that are medication-dosed that dissolve in the mouth similar to the breath strip. Typical medication-dosed films are disclosed in U.S. Pat. Nos. 7,434,692 and D533,463. The breath strip of this invention can include and oral medication. However, that is not necessary for the removal of a bad breath.

Generally speaking, a pressure sensitive inner adhesive surface secures a packet or breath strip assembly that includes the breath strip. A variety of pressure sensitive adhesives and construction patterns are disclosed in the industry. Typical patents showing adhesive patterns and amounts to be used include U.S. Pat. Nos. 6,730,396; and D469,120.

A need exists to provide a breath strip with the purchase of any drink that can result in the user obtaining an objectionable breath or taste when the drink is consumed. The breath strip should be attached to the drink container or a sleeve on the container to be accessed by the consumer after consuming the drink.

Accordingly, an object of the present invention is to provide a breath strip which is immediately available to a person having consumed a beverage from a container which causes the person to have a objectionable breath odor or a bad taste in their mouth.

Another object of the present invention is to attach a breath strip assembly to a drink container or a sleeve on the container so the consumer purchases the drink and the breath strip at the same time.

Yet another object of the present invention is to provide different flavors of soluble film breath strips that can be selected and included with the purchased of a drink.

A further object of the present invention is to provide a pouch associated with a drink container for holding a breath strip which is easily accessible by entry into the pouch.

### SUMMARY OF THE INVENTION

Some of the above objectives are accomplished according to the present invention by providing a system for treating the symptoms of a user having a bad breath or taste after drinking a liquid substance. The invention comprises a drink container initially containing the liquid substance consumed by the user. A breath strip assembly forming a strip pouch is carried by the drink container. A single soluble film breath strip is housed in the strip pouch to be accessed by the user and dissolved in the mouth to remove the users bad breath and taste.

In one aspect of the invention the breath strip assembly comprises a flexible substrate having front and rear surfaces. The substrate is attached to the drink container using a pressure sensitive rear adhesive between the rear surface and the drink container. A removable film is partially attached to the front surface of the substrate using a pressure sensitive front adhesive on the substrate in a pattern to provide the strip pouch. A peel tab of the film is used to remove the film from the substrate to access the breath strip.

In another aspect of the invention the pressure sensitive rear adhesive is initially protected by an adhesive protector. The adhesive protector is removed prior to attaching the breath strip assembly to the drink container.

In other aspects of the invention the breath strip assembly is heat and moisture resistant, biodegradable and flexible to be formed around the drink container.

In a second embodiment of the invention the drink container further includes a sleeve around the container for the user to grasp when holding the container. The breath strip assembly is carried directly by the sleeve of the drink container in lieu of attaching the assembly directly to the drink container.

In a third embodiment of the invention the flexible substrate is not used and the removable film is attached directly to the drink container or the sleeve. A removable film is partially attached to the drink container or the sleeve using a pressure sensitive rear adhesive on the film in a pattern to provide the strip pouch. A peel tab of said removable film is used to remove said removable film from the drink container or the sleeve to access said breath strip.

In another aspect of the invention a method is provided for treating the symptoms of a user having a bad breath or taste after drinking a liquid substance from a drink container. The method comprises the first step of providing a breath strip assembly having a flexible substrate with a removable film attached to a front surface of said substrate using a pressure sensitive adhesive in a pattern to form a strip pouch that contains a single soluble film breath strip. A second step includes coating a rear surface of the breath strip assembly with a pressure sensitive rear adhesive. The third step includes attaching the breath strip assembly to the drink container prior to using the container for dispensing the liquid substance to the user. A fourth step includes removing the removable film to access the breath strip in the strip pouch after consuming at least part of the liquid substance. The fifth step includes the user placing the breath strip in the mouth to remove the bad breath and taste caused by consuming the liquid substance from the drink container.

In a further aspect of the method, the step of attaching the breath strip assembly to the drink container includes the step of initially providing an adhesive protector to protect the breath strip assembly from attachment to other objects prior to attachment to the drink container and the step of removing the adhesive protector prior to attaching the breath strip assembly to the drink container.

In another aspect of the method the flexible substrate is not provided in keeping with the third embodiment of the invention. This method comprises the first step of providing a breath strip assembly having a removable film to be attached to an outer surface of the drink container using a pressure sensitive adhesive in a pattern to form a strip pouch containing a single soluble film breath strip. A second step includes attaching the breath strip assembly to a drink container prior to using said container for dispensing the liquid substance to the user. In a third step the method again includes removing the removable film to access the breath strip in the strip pouch after consuming at least part of the liquid substance. The fourth step again includes placing the said breath strip in the user's mouth to remove the bad breath and taste caused by consuming the liquid substance from the drink container.

Additional applications are also realized within the scope of this invention. Applications include providing a breath strip readily available for the treatment of a bad breath obtained by consuming a drink, as well as the removal of a bad taste acquired by the user.

### DESCRIPTION OF THE DRAWINGS

The construction designed to carry out the invention will hereinafter be described, together with other features thereof.

The invention will be more readily understood from a reading of the following specification and by reference to the accompanying drawings forming a part thereof, wherein an example of the invention is shown and wherein:

FIG. 1 is a perspective view of a drink container with an attached breath strip assembly of this invention containing an easily accessible breath strip;

FIG. 2 is a front elevation view of the breath strip assembly of this invention showing the printed information on the assembly;

FIG. 3 is a rear elevation view of the breath strip assembly of FIG. 2 showing adhesive protectors being removed from the assembly so that the assembly can be attached to the drink container by a rear adhesive;

FIG. 4 is a partial perspective view of the invention of FIG. 1 showing a removable film being removed from the flexible substrate by two fingers on a hand of the user to obtain access to the breath strip in the strip pouch;

FIG. 5 is a cross-sectional view of the breath strip assembly of FIG. 2 taken along line 5-5 in FIG. 2;

FIG. 6 is an enlarged view of a portion of the cross-section of FIG. 5 taken for the area defined by the circle 6 in FIG. 5;

FIG. 7 is a partial cross-sectional view of the invention of FIG. 1 taken along line 7-7 in FIG. 4;

FIG. 8 is a perspective view of a second embodiment of the invention of FIG. 1 including a sleeve around the container with the breath strip assembly attached to the sleeve;

FIG. 9 is a partial perspective view of the invention of FIG. 1 showing a removable film being removed directly from the drink container by two fingers on a hand of the user to obtain access to the breath strip in the strip pouch; and

FIG. 10 is a partial cross-sectional view of the invention of FIG. 1 taken along line 10-10 in FIG. 9.

### DESCRIPTION OF A PREFERRED EMBODIMENT

Referring now in more detail to the drawings, the invention will now be described in more detail. In a first embodiment the invention includes a breath strip assembly or packet attached to a drink container. The breath strip assembly has a removable film attached to a front side of a flexible substrate using a pressure sensitive adhesive in a pattern to create a strip pouch isolated from the environment in which to house a soluble film breath strip. The assembly is attached to the drink container using a pressure sensitive adhesive on a rear surface of the flexible substrate. The user has immediate access to the breath strip after consuming a liquid substance resulting in the user having a bad breath or an objectionable taste.

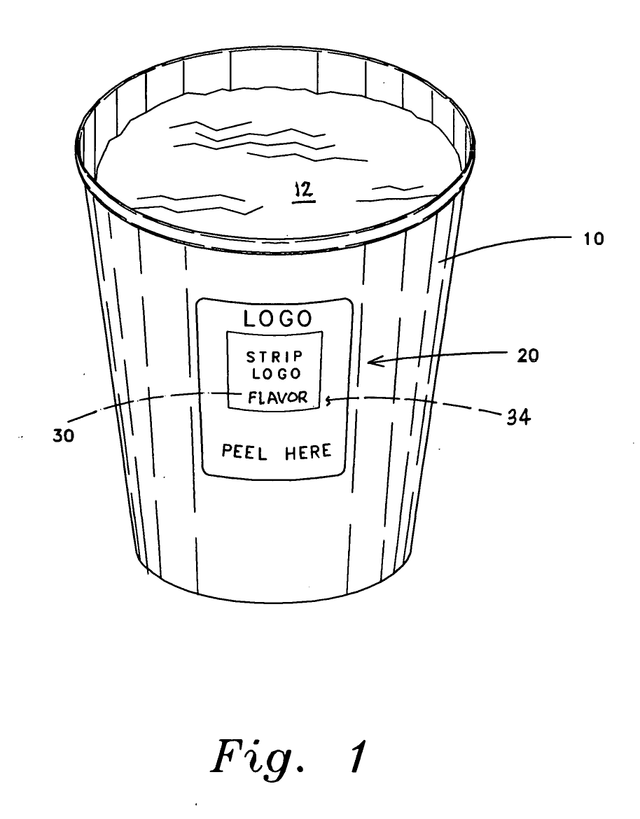

A breath strip assembly 20 attached to a drink container 10 is illustrated in the perspective view of FIG. 1. The drink container is filled with a liquid substance 12 to be consumed by a user. The liquid substance can be any substance that alters the breath of the user; to the extent that it may be objectionable to the user or others in close contact with the user. It also may be any liquid substance that leaves the user with an objectionable taste in their mouth. A typical liquid substance having these features is coffee. The breath strip assembly has a strip pocket 34 that provides moisture barrier properties. A breath strip 30 is to be accessed when the user has consumed at least part of the liquid substance. Information on the breath strip assembly and its proper use can be printed directly on the assembly, as illustrated along with a logo.

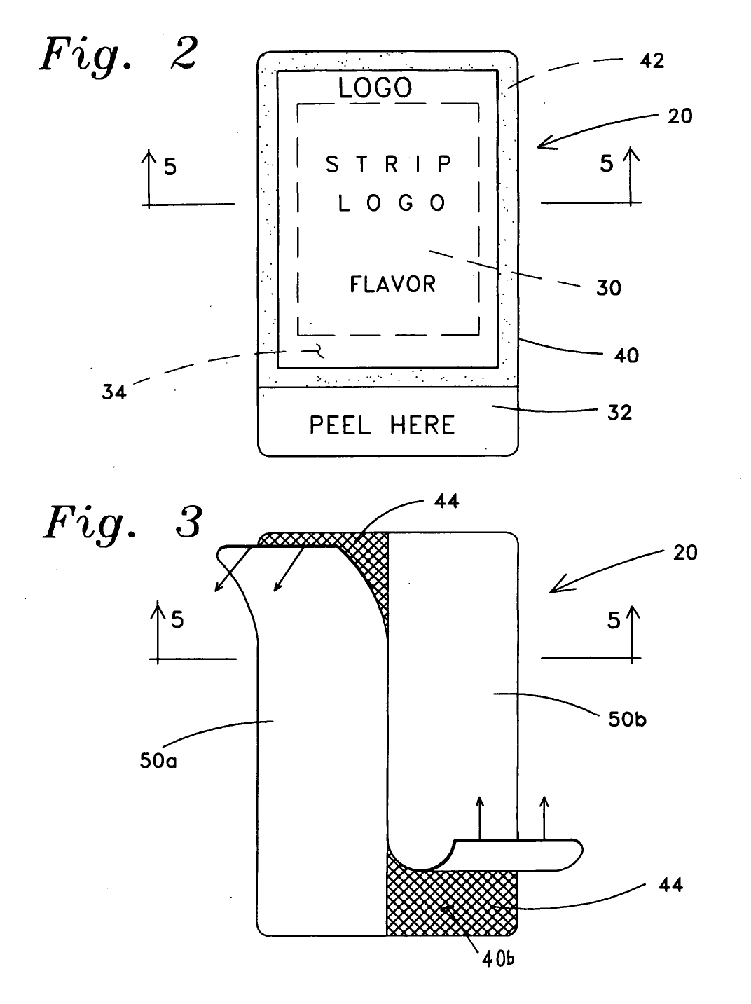

The laminated breath strip assembly is described in more detail by referring to the illustrations of FIGS. 2 and 3. The front elevation view of FIG. 2 shows strip pocket 34 formed by a removable film 32 partially attached to a flexible substrate 40. The substrate is relatively rigid but flexible to be formed around the surface of the drink container. Preferably, the removable film is transparent. The strip pocket is formed by a front adhesive 42 on the flexible substrate in a pattern to form the strip pouch. The location of the front adhesive is illustrated by the stippled area of FIG. 2. Preferably, the front adhesive is placed on front surface 40a of the flexible substrate when laminating the breath strip assembly. However, the front adhesive can be placed on the rear side of the removal strip within the scope of this invention. (see the third embodiment discussion below). Breath strip 30 is placed within the strip pouch to be accessed by the user by peeling back removable film 32 from the flexible substrate. Assistance can be given to the user by labeling that portion of the removable film not attached to the flexible substrate as “PEEL HERE”. Information about the breath strip's use and manufacturer (including logos) can be printed directly on the removable film or the flexible substrate, if the removable film is transparent, to eliminate added labels.

The rear elevation view of breath strip assembly 20 is shown in FIG. 3. A rear surface 40b of the flexible substrate has a pressure sensitive rear adhesive 44 applied over a large portion (or all) of the rear surface of the flexible substrate. Generally speaking, the breath strip assembly will be attached to the drink container by the manufacturer of the drink container. However, it can be added to the drink container at the point-of-sale of the drink. Therefore, rear adhesive 44 must be protected until used to attach breath strip 20 assembly to drink container 10. Adhesive protectors 50a and 50b are added to rear surface 40b. Preferably, two adhesive protectors are used so they can be easily removed (see FIG. 3). Alternately, only one adhesive protector the size of the flexible substrate can be used. Two adhesive protectors rather than a single one are preferred to make their removal easier.

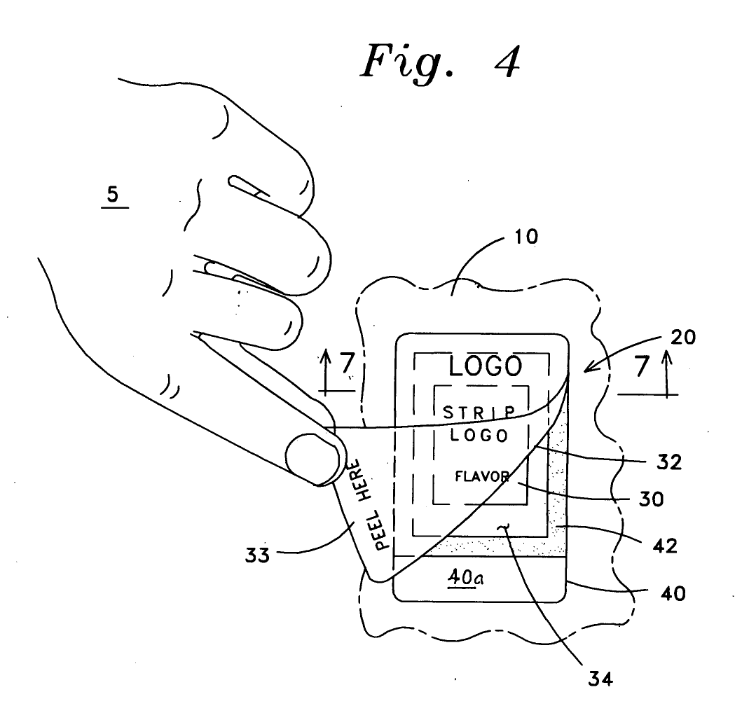

An illustration of a user accessing breath strip 30 in strip pocket 34 is shown in FIG. 4. Two fingers of a hand 5 of the user is peeling away removable strip 32 from flexible substrate 40. The flexible substrate remains attached to drink container 10. Removal is made by gripping the removable strip at peel tab 33 between the index finger and the thumb adjacent the PEEL HERE location. The adhesive strength of front adhesive 42 is not strong enough to keep the removable film attached to front surface 40a of the flexible substrate; so that film 32 is removed to the extent that breath strip 30 can be removed from strip pouch 34. The user places the breath strip in his or her mouth to remove the odor and/or taste of the liquid substance consumed from the drink container.

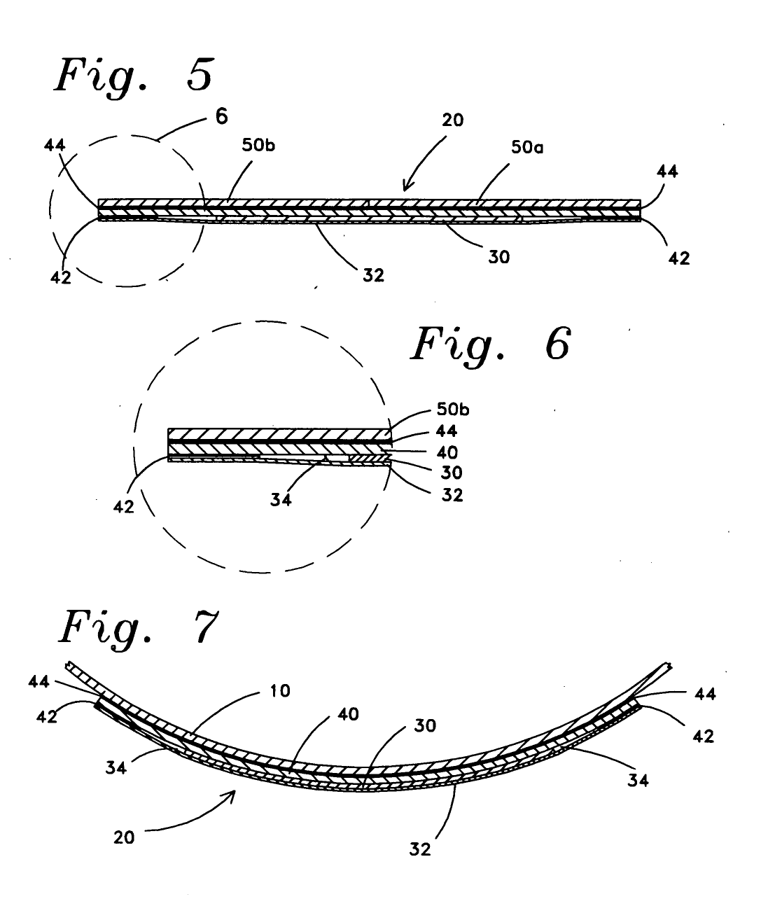

The breath strip assembly is laminated together and attached to the drink container. The cross-sectional views of FIGS. 5 and 6 illustrate how breath strip assembly 20 is laminated. The cross-sectional view of FIG. 7 illustrates how the breath strip assembly is deformed and attached to the drink container. First, consider the breath strip assembly alone in FIGS. 5 and 6. The objective of the lamination is to provide strip pocket 34 for breath strip 30. If necessary, the materials can have moisture barrier properties and heat resistant capabilities. Flexible substrate 40 forms a relatively rigid and flat foundation for the strip pouch. Removable film 32 is a thin material preferably the same size as the flexible substrate. The removable film is partially attached to the flexible substrate by a pressure sensitive front adhesive 42 in a pattern to provide the strip pouch. The breath strip can move freely in the strip pouch.

As a result of the flexible substrate being deformable, the breath strip assembly can be formed to the curvature of the drink container, as illustrated in FIG. 7. After removing the adhesive protectors, breath strip assembly 20 is pressed onto the side of drink container 10. The pressure sensitive rear adhesive 44 secures the remaining breath strip assembly to the drink container. Breath strip 30 is now accessible in strip pouch 34 by removing removable film 32.

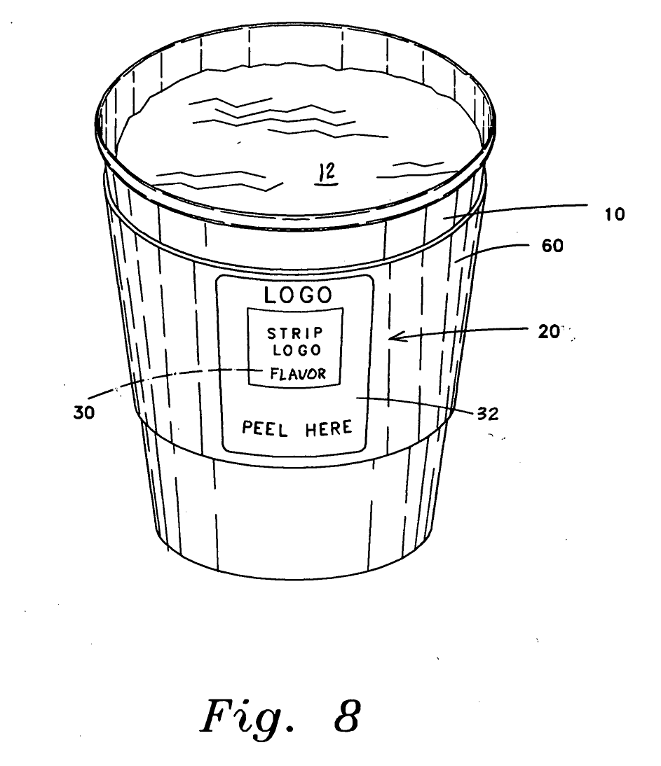

In a second embodiment of the invention a sleeve 60 is provided to protect the user from a very hot or cold liquid substance in the drink container. In this configuration, breath strip assembly 20 is placed directly on the sleeve, as illustrated in FIG. 8. The sleeve is a commonly used item by retailers selling a hot liquid, such as coffee. Retailers include McDonalds®, Starbucks® and the like. Strip pouch 34 is realized by attaching the breath strip assembly directly on the sleeve. A pressure sensitive adhesive placed on rear surface 40b of the flexible substrate is again used to attach the breath strip assembly to the front side of the sleeve; either by the sleeve manufacturer or at the point-of-sale of the sleeve retailer.

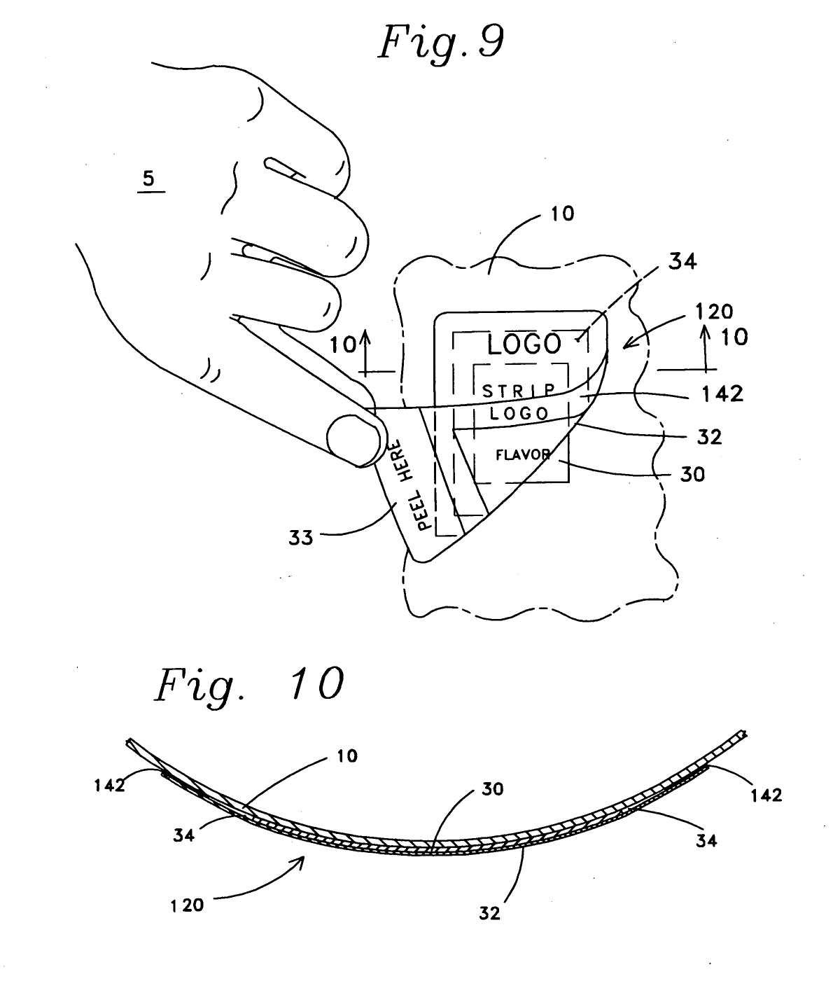

In a third embodiment of the invention breath strip assembly 120 is simplified by removing the flexible substrate; so removable film 32 can be attached directly to drink container 10 to form strip pocket 34, as illustrated in FIG. 9. A pressure sensitive adhesive 142 is placed on the back side of the removable film in the same pattern as previously used on the front surface of the flexible substrate. The cross-sectional view of FIG. 10 illustrates how breath strip assembly 120 having only removable film 32 is deformed and attached to the drink container. This embodiment of the invention is applicable when the heat resistant and moisture barrier properties of the breath strip assembly are not important. In addition, the relative cost of breath strip assembly 120 can be improved if the flexible substrate can be eliminated. Drink containers 10 can be supplied by the manufacturer that include this simplified breath strip assembly.

In the third embodiment of the invention the flexible substrate can also be replaced by the sleeve. The strip pouch can be realized by attaching removable film 32 directly on sleeve 60. This configuration again uses the simplified breath strip assembly where the flexible substrate has been eliminated. A pressure sensitive adhesive placed on the back side of the removable film is used in the same pattern as previously used on the front surface of the flexible substrate (see FIG. 3) to create strip pouch 34. Any indicia can also be placed on the removable film, the sleeve or the drink container. Preferably the removable film is placed on the sleeve by the sleeve manufacturer for this embodiment. A typical sleeve is that disclosed in U.S. Pat. No. 6,863,644 issued 8 Mar. 2005, which is incorporated herein by reference hereto.

Preferably, flexible substrate 40 is a plastic substrate made of a thin plastic material such as polystyrene, polyethylene, polypropylene of other polymeric of plastic type material. The plastic substrate has sufficient thickness and flexibility to form and hold the breath strip assembly on the drink container. Alternately the flexible substrate can be a paperboard with a moisture barrier coating applied to the front side. The coating can be a carboxylated amine selected from the group consisting of a vinylidene chloride polymer, a vinylidene dichloride polymer and a vinylidene chloride/vinylidene dichloride copolymer emulsion. In addition the flexible substrate can be a metal foil, such as an aluminum foil, to help in providing heat resistance to the breath strip assembly.

The removable film can comprise any desired type of foils, nylon, co-extruded films or other film. Preferably, the removable film is polyethylene BAREX® of BP Chemicals, Inc. of Naperville, Ill.

For pressure sensitive adhesives 42 and 44 a variety of commonly available pressure sensitive adhesives can be used. The specific choice generally depends upon the desired conditions of application. Usable adhesives include those available from H. B. Fuller of St. Paul Minn.; Ashland Petroleum of Columbus, Ohio; and Hartland Adhesives of New Berlin, Wis. Typically the amount of adhesive applied per unit area as front adhesive 42 is about 35% to about 65% of the amount per unit area applied as rear adhesive 44. This allows the removable film to be removed without pulling the flexible substrate from the drink container.

A silicone coated paper can be used for adhesive protectors 50a and 50b on the rear side of prior to applying the breath strip assembly to the drink container. A utilizable material for the adhesive protectors is a 43 lb. diversified Kraft paper with a silicone release coating such as that available from the Siltech Division of Technicote Company of Miamisburg, Ohio.

Material used for drink container 10 can be any variety of commonly available paper or plastic material known in the industry. The hot drink paper cup is commonly used for those liquid substances which are served hot. Plastic type containers are generally used for cold drinks.

The soluable film breath strips 30 are food grade and edible. Edible films include pullulan and antimierobially effective amounts of the essential oils thymol, methyl salicylate, encalyptol and menthol. There are a number of flavors available that include chlorophyll and peppermint, lemon cough drop, sweet mint caffeine and spearmint. Ingredients that produce these different flavors may include various amounts of sodium alginate, gum arabic, glycerin, maltic acid, chlorophyll, sucrolase, aspartame, acesulfame K, caffeine, propylene glycol, neatame, menthol and coloring and flavoring agents. These and others are produced by Watson's Film Technology Division of West Haven, Conn. The preferred flavor for the present invention is peppermint.

While a preferred embodiment of the invention has been described using specific terms and a particular prior art reference, such description is for illustrative purposes only, and it is to be understood that changes and variations may be made without departing from the spirit or scope of the following claims.

What is claimed is:

1. A system for treating the symptoms of a user having a bad breath or taste after drinking a liquid substance comprising:
a drink container initially containing the liquid substance consumed by the user;
a breath strip assembly forming a strip pouch carried by said drink container; and
a single soluble film breath strip housed in said strip pouch to be accessed by the user and dissolved in the mouth to remove the users bad breath and taste.

2. The system of claim 1 wherein said breath strip assembly comprises:
a flexible substrate having front and rear surfaces, said substrate being attached to said drink container using a pressure sensitive rear adhesive between said rear surface and said drink container; and
a removable film partially attached to said front surface of said substrate using a pressure sensitive front adhesive on said substrate in a pattern to provide said strip pouch, wherein a peel tab of said film is used to remove said film from said substrate to access said breath strip.

3. The system of claim 2 wherein said pressure sensitive rear adhesive on said flexible substrate is initially protected by an adhesive protector which is removed prior to attaching said breath strip assembly to said drink container.

4. The system of claim 2 wherein said breath strip assembly is both heat resistant and provides a moisture barrier to keep said breath strip from being dissolved or reduced in effective strength.

5. The system of claim 2 wherein said breath strip assembly has indicia printed thereon to provide the user with at least, but not limited to, instructions in accessing the breath strip as well as a flavor of the breath strip and a logo.

6. The system of claim 2 wherein said breath strip assembly is made of a material which is biodegradable.

7. The system of claim 2 wherein the soluble film breath strip is made to have a flavor of one of either chlorophyll with peppermint, lemon cough drop, sweet mint caffeine or spearmint.

8. The system of claim 2 wherein said breath strip assembly is made to be deformable to conform to the shape of the drink container.

9. The system of claim 1 wherein said breath strip assembly comprises:
a removable film partially attached to said drink container using a pressure sensitive rear adhesive on said removable film in a pattern to provide said strip pouch; and
said removable film having a peel tab which the user grabs with two fingers to remove said film from said drink container to access said breath strip.

10. The system of claim 1 wherein said drink container further includes a sleeve around said container for the user to grasp when holding said drink container, wherein the breath strip assembly is carried directly by said sleeve of said drink container, and wherein said sleeve provides heat resistant properties and a moisture barrier for said breath strip.

11. The system of claim 10 wherein said breath strip assembly comprises:
a flexible substrate having front and rear surfaces, said substrate is attached to said sleeve of said container using a pressure sensitive rear adhesive between said rear surface of said flexible substrate and said sleeve; and
a removable film partially attached to said front surface of said substrate using a pressure sensitive front adhesive on said flexible substrate in a pattern to provide said strip pouch, wherein a peel tab of said film is used to remove said film from said sleeve to access said breath strip within said pouch.

12. The system of claim 11 wherein said pressure sensitive rear adhesive of said flexible substrate is initially protected by an adhesive protector which is removed prior to attaching said breath strip assembly to said sleeve.

13. The system of claim 10 wherein said breath strip assembly comprises:
a removable film partially attached to said sleeve of said container using a pressure sensitive rear adhesive on said film in a pattern to provide said strip pouch; and
a peel tab of said removable film used to remove said removable film from said sleeve to access said breath strip.

14. A method for treating the symptoms of a user having a bad breath or taste after drinking a liquid substance from a drink container comprising the steps of
a) providing a breath strip assembly having a flexible substrate with a removable film attached to a front surface of said substrate using a pressure sensitive adhesive in a pattern to form a strip pouch containing a single soluble film breath strip;
b) coating a rear surface of said flexible substrate with a pressure sensitive rear adhesive;
c) attaching said breath strip assembly to the drink container prior to using the container for dispensing the liquid substance to the user;
d) removing said removable film to access said breath strip in said strip pouch after consuming at least part of the liquid substance; and
e) placing said breath strip in the user's mouth to remove the bad breath and taste caused by consuming the liquid substance from said drink container.

15. The method of claim 14 where the step of attaching said breath strip assembly to said drink container further comprises the steps of:
a) providing an adhesive protector to initially protected said breath strip assembly from sticking to other objects prior to attachment to the drink container; and
b) removing said adhesive protector prior to attaching said breath strip assembly to said drink container.

16. The method of claim 14 wherein the step of using a drink container includes the step of providing a sleeve around said drink container as a part of said drink container for grasping and holding said container.

17. A method for treating the symptoms of a user having a bad breath or taste after drinking a liquid substance from a drink container comprising the steps of:
a) providing a breath strip assembly having a removable film to be attached to an outer surface of the drink container using a pressure sensitive adhesive in a pattern to form a strip pouch containing a single soluble film breath strip;
b) attaching said breath strip assembly to a drink container prior to using said container for dispensing the liquid substance to the user;
c) removing said removable film to access said breath strip in said strip pouch after consuming at least part of the liquid substance; and
d) placing said breath strip in the user's mouth to remove the bad breath and taste caused by consuming the liquid substance from said drink container.
